(19) United States
(12) Patent Application Publication
(10) Pub. No.: US 2026/0199160 A1
(43) Pub. Date: Jul. 16, 2026

(54) A METHOD FOR REDISTRIBUTION OF BODY PRESSURE DISTRIBUTION BY A SUPPORT DEVICE AND THE SYSTEM THEREOF

(71) Applicant: EBIO TECHNOLOGY INC, NEW TAIPEI CITY (TW)

(72) Inventors: DER-CHIH SHR, New Taipei City (TW); DE-FEN SHIH, New Taipei City (TW); DE-HUI SHIH, New Taipei City (TW)

(21) Appl. No.: 19/135,900

(22) PCT Filed: Feb. 1, 2023
(86) PCT No.: PCT/CN2023/074114

§ 371 Date: Jun. 5, 2025

(51) Int. Cl. A61G 7/057; G16H 50/30

(52) U.S. Cl. CPC A61G 7/05776; G16H 50/30; A61G 2203/34

(57) ABSTRACT

The present invention focuses on the pressure injury appeared on one or more bony processes of the human body. When the user's body is supported by a support device such as a mattress, the body posture is acquired by referring to the measured two-dimensional pressure distribution. Next, one or more bony processes of the human body are identified by referring to the user's pathological information. Then, by analyzing the pressure injury probability and corresponding pressure of each bony process, the pressure applied by the support device to different parts of the user's body are adjusted. Thus, the risk of the pressure injury on all bony pressures are decreased.

### BACKGROUND OF THE INVENTION

### 1. Field of the Invention

The present invention is about a method and system for redistribution of body pressure by support device, which can improve the shortcomings of conventional pressure sore prevention mattress, which cannot accurately predict the higher risk areas of pressure injury in human body and deal with them effectively.

### 2. Description of the Prior Art

Although directly reducing the pressure on the body's more pressurized areas can reduce the chance of developing Bedsore, one or more areas of the body that are more susceptible to pressure injury are not exactly the same as one or more areas of the body that are more pressurized, such as the buttocks. For example, the buttocks are often not a high-risk area for pressure ulcers, but the coccyx, located at the top 10 centimeters above the buttocks, is often a high-risk area for pressure ulcers. In other words, the human body is born with an uneven distribution of apophysis position, so the ability to withstand critical pressure will vary from location to location, with thin skin and bones being less tolerant and thicker skin and flesh being more tolerant, resulting in specific areas being at high risk for pressure sores. Therefore, existing commercial products and related technological developments often do not specifically address one or more areas of the body that are more susceptible to pressure injuries. This is because when a user is lying or sitting or lying on the support device, the user's body is often under greater pressure on the parts of the body that are in direct contact with the support device, such as the buttocks, thighs, calves, shoulders and abdomen, where there are more muscles, fat and subcutaneous tissues, which may be able to cushion the impact of pressure on blood circulation and body tissues. It may also be able to transfer the pressure on this area to nearby body parts, thus reducing the chance of injury caused by continuous pressure or sudden exposure to greater pressure.

In contrast, one or more apophysis position of the human body are almost directly covered by the skin, and there are less muscle, fat and subcutaneous tissue between the skin and the bone, and there is also a lack of blood vessels to fully carry out blood flow. Circulation, so that the pressure on the apophysis position cannot be buffered or transferred, and it is more vulnerable to injury than other body parts when subjected to the same pressure. Suffer from sustained stress or short-term intense stress. That is, apophysis positions are often prone to pressure injuries, even if they are not the most stressed parts of the user's body. Therefore, the existing method of directly reducing the pressure on the parts subject to higher pressure (such as adjusting the air volume of the air bag to change the pressure on the human body in contact with the air bag) basically cannot effectively improve the pressure on the apophysis position of the human body that is prone to pressure. Injury issues, even though these apophysis positions are often adjacent to these stressed body parts. FIG. 1A shows the coordinate group of high-risk parts of the apophysis position where the user's body is in direct contact with the support device in the body posture (supine position, lateral position and half-sleeping position).

The internal pressure of the airbag is generally known to be in a uniform pressure state, but the external surface reaction pressure caused by the external force applied to the airbag is not uniformly distributed, and will vary with the shape of the intersection of the force applying object and the surface of the airbag. It is different, as shown in FIG. 1B as an example, even if a disc object and a conical object of the same weight act on the same airbag, the resulting local pressure will also present a different reaction pressure distribution, and the greater the shape fluctuation, The resulting uneven pressure will also become more serious, as shown in FIG. 1C.

The human body has the appearance of high and low, rather than flat structure, therefore, when the human body lies on the soft mattress, the rebound force and tension of the mattress body will certainly make the reaction pressure of the body pressure with the high and low undulation of the mattress and the softness and hardness present different degrees of uneven pressure distribution, as shown in FIG. 1C. When this mattress is mainly structured with airbags of several divisions, the pressure change of the airbag filling in each area will change the shape and softness and hardness of this bed body. The saturation pressure mentioned in general is often misunderstood as the air pressure inside the airbag, and the saturation pressure is the saturation pressure inside the airbag, and the actual source of pressure is twofold: one is the body pressure, and the other is the internal pressure of the airbag.

Therefore, when the pressure adjustment of the airbags in individual zones is carried out, it will be linked to the overall surface action and reaction pressure distribution. Take FIG. 1C as an example, the airbags in P1~P6 area are inflated with equal pressure, which will present a very high surface pressure distribution outside the hip area. This is because the total weight of the body is fixed and the support force distribution in one area rises, which inevitably causes the support force in other areas to fall, forming a new balance. It can be seen from the physical principle that the air mattress can redistribute the local pressure distribution pattern of lying body pressure action and reaction force by adjusting the airbag pressure of each zone. The traditional intuition holds that the air mattress can directly increase or decrease the reaction pressure on the surface of the human body by simply charging and discharging the air pressure in the airbag. This method will not make meaningful decompression work on the actual surface pressure of the human body. The existing technologies all focus on the adjustment of a single airbag, that is, first calculate the single part of the human body where pressure injury may occur, and then adjust the internal airbag pressure of the single airbag corresponding to the part. However, no matter how well this method analyzes the fine enough damage location, the relative method that it can deal with but still can only be handled simply and simply with the internal air pressure adjustment of the corresponding single air bag, it is impossible to effectively achieve the means of adjusting relative risk positions.

There is an urgent need to develop a method and system for redistributing body pressure distribution through support device in related industries at present, new methods and systems for reducing or even eliminating the risk of pressure injuries to the user's body.

### SUMMARY OF THE INVENTION

Different from the traditional method of directly inflating and deflating the airbags, when regulating the pressure distribution of the human body, it is necessary to correctly consider and calculate all the pressure distribution of the human body covered in the air mattress and the interaction of the airbags in each area. Know how to redistribute and combine the internal gas pressure of all airbags in each zone, and finally make the reaction pressure on the human body lying be redistributed evenly or achieve other desired distribution forms to eliminate the pressure on bedridden patients Risk of epidermal pressure injury. One purpose of the present invention proposes a method for redistribution of body pressure distribution by a support device: first, when the user is supported by a support device, such as when sitting or lying on a mattress, a plurality of pressure sensors in the support device are used to measure and generate a two-dimensional pressure distribution corresponding to the pressure exerted by the human body on the support device. Then, analyze the two-dimensional pressure distribution to calculate the current body posture of the user. That is to say, how the musculoskeletal of the user's body are distributed on the support device is estimated from the measured pressure distribution, the characteristic parameters of this pressure distribution can be calculated to judge the user's current posture. Then, analyze the body posture, and mark the respective positions of one or more apophysis position of the user's body on the support device, that is to say, it is not to find out one or more places where the user's body is subjected to greater pressure. Rather, it identifies one or more areas of the user's body that are more likely to be injured by sustained or sudden high pressure. Next, judge whether the occurrence rate of pressure injury corresponding to one or more apophysis position is acceptable. For example, whether the occurrence rate is less than a common critical rate value equal to one of all bony prominences or less than the respective critical rate value of one of the individual bony prominences, if the above conditions are met, then it is acceptable, if both are acceptable, then no further treatment is required, if not all are acceptable, then the support force generated by one or more support units should be cyclically adjusted until the occurrence rate of pressure injury to all bony prominences is acceptable.

The method for redistributing body pressure distribution by a support device proposed by the present invention is different from the existing methods for improving decubitus ulcers. The main difference is that the method proposed by the present invention is to first find out one or more specific positions of one or more apophysis position of the user's body on the support device. And adjust the different supporting forces exerted by the support device on different parts of the user's body as needed, in order to reduce the pressure on the user's body at one or more apophysis position, thereby reducing or even eliminating the probability of pressure injury occurring at these apophysis position. In other words, how to find the location of the apophysis position, how to judge the probability of pressure injury on the apophysis position, and how to adjust the different supporting forces exerted by the support device on different parts of the user's body so as to reduce or even eliminate the apophysis position The probability of pressure injury is the main feature of the method proposed by the present invention.

Another purpose of this invention is to reduce and eliminate pressure injuries that are prone to occur at the apophysis position and to adjust the pressure on each part of the user's body to reduce pressure injuries. That is, propose a method to redistribute the body pressure distribution on the support device, compared with existing commercial products and existing technology research and development, it focuses on decubitus ulcers that are prone to occur in places where the body is subject to greater pressure and directly reduces the pressure on places where the body is subject to greater pressure. The technical characteristics of the present invention are at first to find out the overall two-dimensional pressure distribution of the body surface, then, after judging the body posture of the user's body on the support device, find out the position of the human apophysis position on the support device. It is also necessary to judge the probability of pressure injury at each apophysis position. Then determine how to adjust the pressure applied by the support device on the user's body to reduce the probability of pressure injury at each bony protrusion. That is to say, starting from the user sitting or lying or lying on a support device such as an air bed, how to measure the pressure generated by the mutual contact between the user's body and the support device, how to generate a two-dimensional pressure distribution relative to the user's body, and how to generate the user's body posture from the two-dimensional pressure distribution, etc. can be the same as the existing products/technologies. However, the existing products/techniques directly find one or more parts of the user's body that are subject to greater pressure from the posture of the user's body, and directly adjust the pressure exerted by the support device on one or more parts.

A further purpose of this invention is to provide a method for redistributing body pressure distribution of support device, including obtaining a two-dimensional pressure image, obtaining characteristic parameters from the analysis of the two-dimensional pressure image, and obtaining body shape factors (Height, weight, waist circumference, limb defects), by comparing and judging from the above machine learning and big data, the lying posture and the coordinate position of each apophysis position point can be obtained, and those points obtained from the lying posture will be oppressed (lying upright Different from lying on the side), according to the characteristics of the pressure image and the lying posture, the specific coordinate position of the apophysis position can be calibrated, compared with the clinical database and calculated how much the coordinate pressure of the dangerous part of the bony protrusion should be reduced to be safe, and converted back to the support unit What kind of shape or hardness should it have to meet the pressure redistribution pattern, while driving the support device and feedback the pressure distribution at the same time, repeat the operation until the target pressure is met.

### BRIEF DESCRIPTION OF THE DRAWINGS

FIG. 1A shows the human posture and the corresponding bony prominence;

FIG. 1B and FIG. 1C show the relationship between the distribution of human body pressure and the equal pressure inside the airbag;

FIG. 2A to 2D are schematic diagrams of the method of redistributing body pressure distribution for one of the inventions support device;

FIG. 3A and FIG. 3B show a schematic diagram of the system architecture of one of the inventions support device redistribution of body pressure distribution method;

FIG. 4 shows the basic flow diagram of a method of redistributing the body pressure distribution of the support device proposed by the present invention;

FIG. 5A and FIG. 5B are schematic diagrams of the application of the invention.

### DESCRIPTION OF THE PREFERRED EMBODIMENTS

What is probed into the invention is a method for redistribution of body pressure distribution by a support device. Detail descriptions of the compositions, structures, elements and steps will be provided in the following in order to make the invention thoroughly understood. Obviously, the application of the invention is not confined to specific details familiar to those who are skilled in the art. On the other hand, the common compositions, structures, elements and steps that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

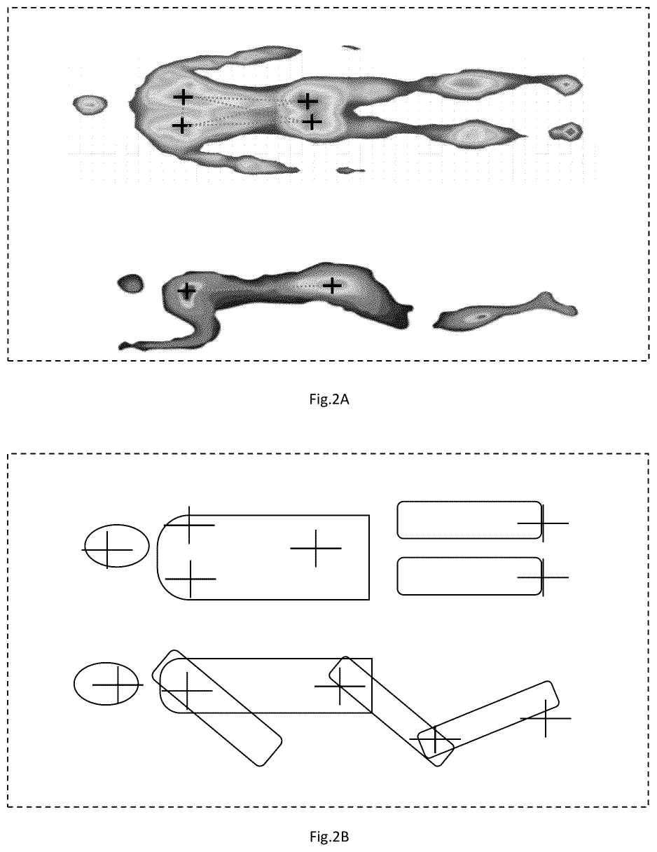

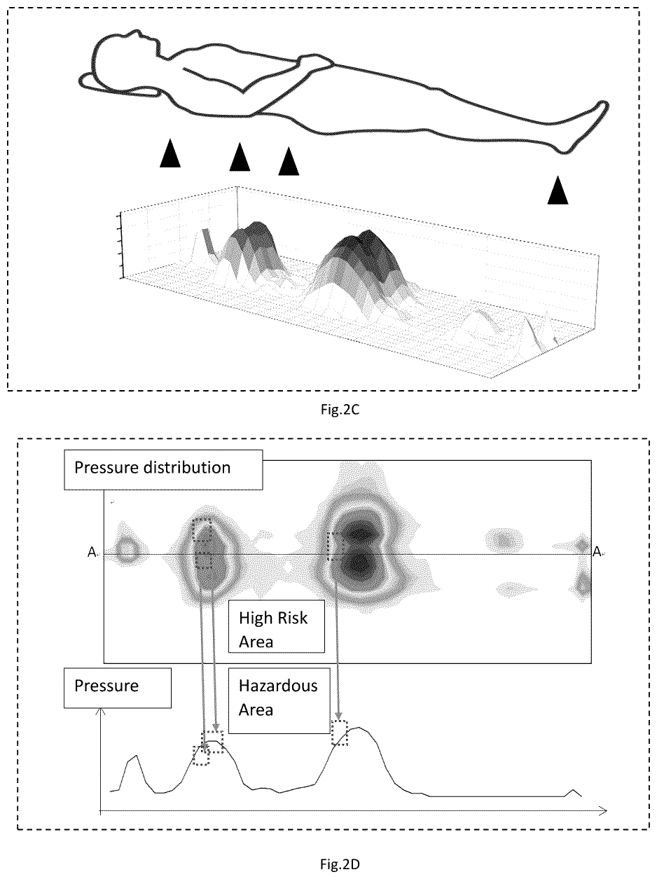

The basic concept of a method for redistributing body pressure distribution by a support device proposed by the present invention is shown in FIGS. 2A to 2D. First, as shown in FIG. 2A, when the user's body is on the support device, the two-dimensional pressure distribution corresponding to the posture of the user's body is obtained by measuring the pressure on different parts of the support device. Taking the user lying upright or sideways on the support device as an example, it can be seen that the parts corresponding to the user's shoulders and buttocks will have greater pressure. Then, calculate the user's body posture based on this two-dimensional pressure distribution, generally speaking, artificial intelligence can be used to calculate, so as to take advantage of the huge computing power of artificial intelligence, the learning ability with more do and more precise. Then, as shown in FIG. 2B, the position of each bony protrusion at the junction of the user's body and the support device is marked out from the calculated user's body posture. In this illustration, the user's body posture is lying or sideways Lie down as an example and mark the apophysis position with a cross. The reason why the body posture needs to be identified first is that the apophysis position where the user's body contacts the support device are not exactly the same under different body postures. For example, when lying on the side, the user's coccyx (coccyx) cannot be in direct contact with the support device, and when lying down, the user's hipbone cannot directly contact with the support device. After the body posture is identified, it is necessary to calculate which apophysis position will connect with the support device, to judge the respective coordinates of these apophysis position on the support device (for example, to calibrate according to the external contour and internal relief features).

As shown in FIG. 2C, the part of the user's body that experiences greater pressure is not necessarily the apophysis position of the user's body (marked with a triangle), although the two are usually adjacent to each other. As shown in FIG. 2D, along the straight line A-A that runs through the middle of the human body from the head to the tail, the pressure on different parts of the human body is different, but one or more parts will always bear the pressure not less than the critical pressure value, from It can be clearly understood in the figure that the pelvis directly corresponds to the place with higher critical pressure (pressure in the high-risk area), but there are also more muscles and fat here to buffer and disperse the pressure, so the peak pressure is not absolutely equal In the actual strained part, this is the safe zone instead. In contrast, a certain segment of the spine that directly bears pressure like skinny (for example, Sacrum Suffering) corresponds to a lower critical pressure and becomes a relatively dangerous area. If the continuous accumulation of pressure exceeds three hours, the pressure of tissue changes will be taken as the critical pressure value for inducing pressure sores. Among them, the critical pressure of pressure injury is related to body shape, posture, and health status, and can be defined by clinical research data or pressure injury literature. It must be noted that the previous technologies all use deduction to directly preset the location of the pressure peak as the strained part, and then directly adjust the air pressure in the airbag where the pressure peak is located. As a result, it is still impossible to effectively alleviate the damage of the actual strained part. Therefore, the present invention proposes that when the user's body weight is fixed, by adjusting the pressure on different parts of the human body, in order to reduce or even eliminate the chance of any part of the body being subjected to pressure above this critical pressure value, that is, pressure reconfiguration is performed. Since the user has a certain body weight, reducing the pressure on one part will inevitably increase the pressure on other parts. Therefore, a major principle for adjusting the pressure on various parts of the human body, that is, after adjustment, the pressure on one or more parts of the human body that are prone to pressure injuries should be lower than the critical pressure value. After adjustment, the pressure on any part of the human body is less than the critical pressure value, which is better. It is not just that after adjustment, various parts of the human body that are greater than the critical pressure value before adjustment can be reduced. When actually adjusting each support unit (such as an airbag) to adjust the pressure on various parts of the user's body, the number of support units that need to be adjusted (such as the number of airbags whose inflation level is adjusted) is generally reduced as much as possible.

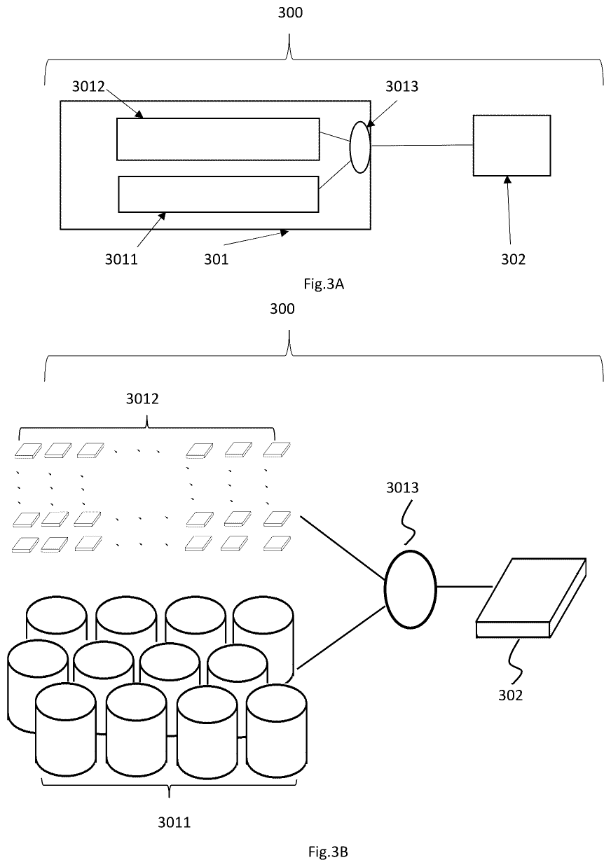

The basic system architecture of the method for redistributing body pressure by the support device proposed by the present invention is shown in FIGS. 3A to 3B. The improved pressure injury system 300 at least includes a support device 301 and a control device 302, and the support device 301 at least includes a plurality of support units 3011, a plurality of pressure sensors 3022 and an interface module 3013. The support units 3011 are located inside the support device 301 and arranged in a two-dimensional array (the first two-dimensional array), and can generate the same or different supporting forces respectively. These pressure sensors 3012 are located between these support units 3011, the support device 301, and specific sides intended to contact the user's body (or to support the user's body), and they are arranged mutually to form a two-dimensional array (second two-dimensional array) inside the support device 301. Thereby, these support units 3011 can respectively generate supporting force to support the user's body sitting or lying or lying on a specific side of the support device 301, and the pressure sensor 3012 can sense the pressure on various parts of the user's body located on a specific side, a two-dimensional pressure distribution corresponding to the posture of the user's body is thereby produced. The interface module 3013 is respectively connected to the support unit 3011 and the pressure sensor 3012, in order to transmit the information used to adjust the support force generated by one or more support units 3011 respectively, and receive the pressure values from the specific side measured by one or more pressure sensors 3012 respectively (that is, the user's body and the support device between pressure values). The interface module 3013 is connected to the control device 302. The supporting force generated by the support unit 3011 is adjusted by the control device 302 according to the measurement result of the pressure sensor 3012. Furthermore, the pressures received by different parts of the entire user's body located on a specific side are adjusted respectively. The control device 302 can analyze the two-dimensional pressure distribution and in order to calculate the user's body posture, further marking the location of one or more apophysis position of the user's body on the specific side. When the control device 302 has an unacceptable probability of pressure injury at at least one condyle, the at least one supporting force generated by the at least one support unit is cyclically adjusted until the probability of pressure injury occurring at one or more apophysis position where the user's body contacts with the support device is acceptable.

The control device 302 can interact with the support device 301 by the interface module 3013, such as receiving measurement data from the pressure sensor 3012 and controlling the support unit 3011 to adjust the support force applied to different parts of the user's body. The control device 302 can be any electronic device with a built-in application program (App) for interacting with the support device 301, such as a smart phone, a tablet, a notebook computer and a desktop computer, etc. The interface module 3013 can be any wired or wireless communication module, such as cables, Bluetooth modules, Wi-Fi modules, infrared modules and wireless communication modules. Moreover, the support device 301 and the control device 302 can be two hardware bodies separated from each other, or two hardware bodies integrated together, for example, one control device 302 corresponds to a plurality of support devices 301 to simplify the work of caring for a plurality of devices at the same time.

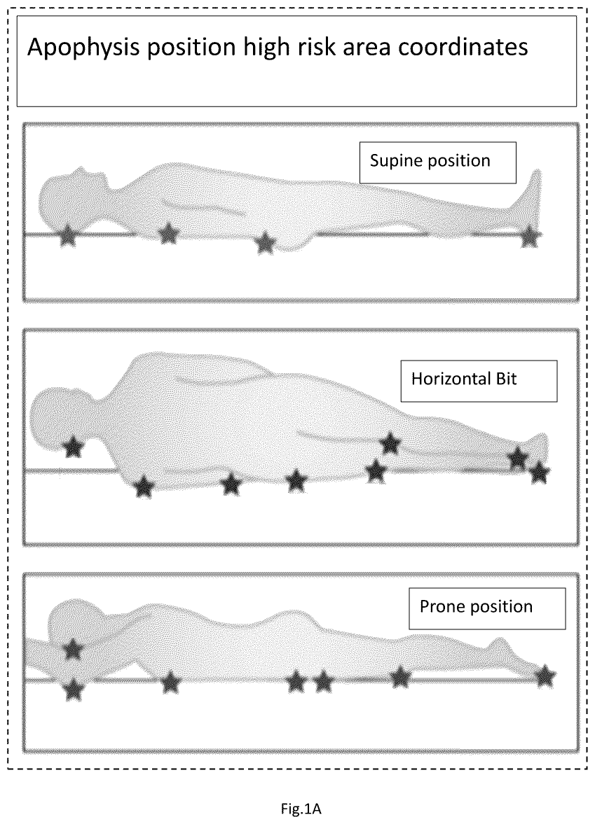

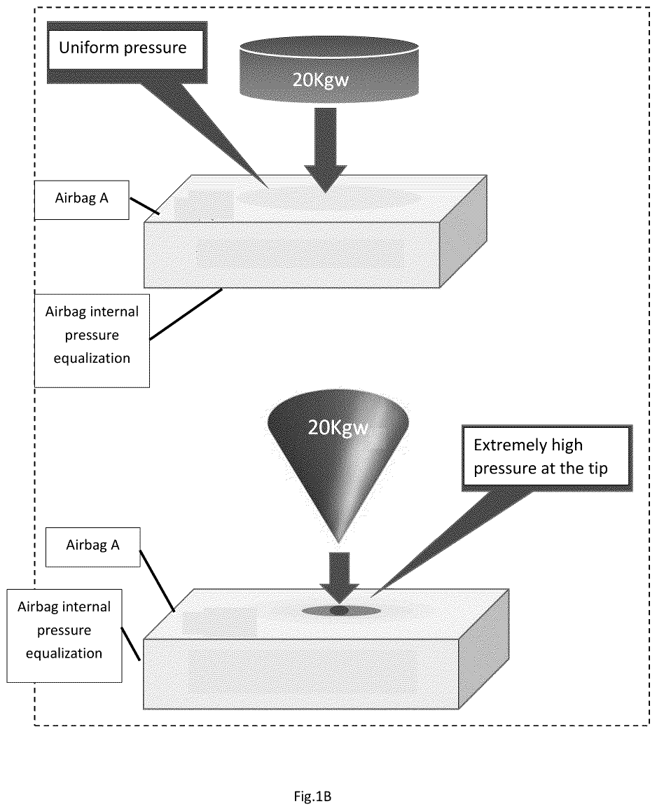

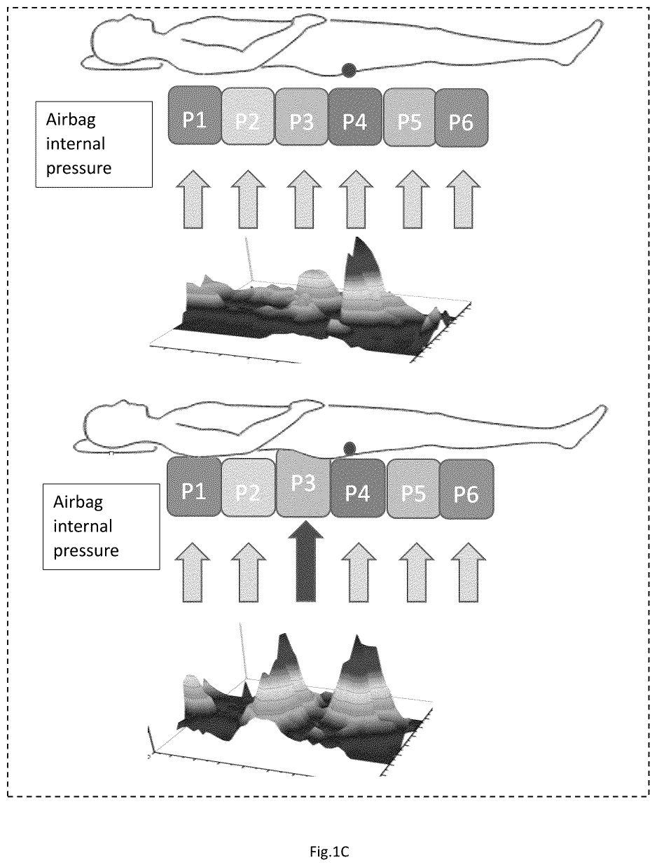

A support device proposed by the present invention to redistribute the body pressure distribution system has the following commonly used options. Since the present invention aims at adjusting the pressure on the bony protruding place of the human body to reduce and eliminate the probability of pressure injury occurring at the bony protruding place, and as previously shown in FIG. 1B, the area of any human apophysis position is often not much different from the size of a coin. Therefore, in order to accurately locate the position of each bony protrusion on the support device 301, the distance between adjacent pressure sensors 3012 often cannot be several integer a plurality of the coin size. Some test results have shown that the distance between adjacent pressure sensors can be kept less than three centimeters, for example, the distance between the edges of each other is less than three centimeters, or the distance between the centers of each other is less than three centimeters. The support unit 3011 can adjust the airbag of the supporting force generated by adjusting its inflation degree, considering the size of the airbag and the pressure sensor 3012. The distribution density 3012 of these pressure sensors is generally higher than the distribution density of the support units 3011, and a plurality of airbags with smaller sizes and more densely arranged with each other can also be used as the support units 3011. In addition, in order to effectively measure the degree of contact between different parts of the user's body and different parts of a specific side of the support device 301 (indicated by the pressure received) when the user is in contact with the support device 301. Since the body posture of the user appearing on the specific side of the support device 301 can be changed at any time. Therefore, the pressure sensors 3012 are often arranged in a two-dimensional array (the first two-dimensional array). The support units 3011 are usually arranged in another two-dimensional array (second two-dimensional array), in order to completely and accurately measure the different pressures caused by different parts of the user's body on a specific side of the support device 301.

In addition, each support unit 3011 can change the supporting force it generates to change the different pressures on different parts of the user's body. Because the outline of the human body does not only have straight edges like a cuboid. Therefore, when the user's body is supported by the majority of support units 3011, different support units 3011 that contact different parts of the user's body 309 often have different adjustable profiles, in order to properly support the user's body and adjust the supporting force exerted on the user's body. That is, the support unit 3011 can adjust its vertical height, hardness or even its horizontal size, and by changing the quantity or flow rate of fluids such as gas or liquid flowing through the interior, the support unit 3011 can change the support force and/or size shape profile. Since the support device 301 is used to reduce the damage to the user's body due to excessive and/or prolonged pressure, the support unit 3011 will generate respective support forces (whether they are the same or different) before the user is on the specific side. Before the user's body is supported by the support device 301, the plurality of airbags as these support units 3011 have been inflated to different heights respectively, in order to properly support the user's body and reduce the discomfort of the user. In addition, the surface of this particular side is covered with a soft or deformable material, in order to relieve the pressure on the user's body when the user's body is in contact with the specific side.

The control device 302 may have a built-in artificial intelligence for processing the information from the pressure sensor 3012 and adjusting the supporting force generated by the supporting unit 3011. The control device 302 can use artificial intelligence to implement the method for improving pressure injury proposed by the present invention, to use various databases and, various reference information and executed tests one by one, continuously optimize this AI and improve pressure injuries more and more accurately. The control device 302 can use artificial intelligence to analyze the two-dimensional pressure distribution measured by the pressure sensors 3012 and calculate the body posture of the user on the support device 301, or use artificial intelligence to calculate the body posture of the user marks the position of one or more apophysis positions of the body on a specific side of the support device 301. The control device can use artificial intelligence to judge the probability of pressure injury at each apophysis position. Also when there is an unacceptable rate of pressure injury to at least one condyle. For example, when they are greater than a common critical probability value for all apophysis positions or are respectively greater than the respective critical probability values for each apophysis position, the supporting force generated by at least one support unit 3011 is cyclically adjusted until the probability of pressure injury occurring at all apophysis positions is acceptable. Alternatively, the artificial intelligence can be trained by referring to the correlation between body posture and the position of one or more apophysis positions obtained by other means. It may also be possible to improve the results of pressure injuries by adjusting one or more support unit s for reference, to train this artificial intelligence.

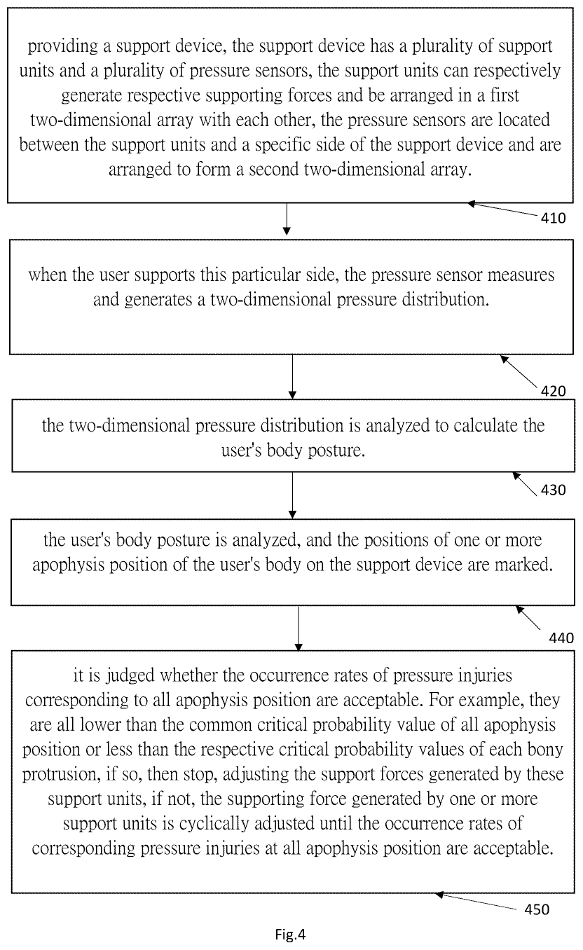

As shown in FIG. 4, the basic flowchart of a system for redistributing body pressure distribution of a support device proposed by the present invention: first, as shown in step 410, providing a support device, the support device has a plurality of support units and a plurality of pressure sensors, the support units can respectively generate respective supporting forces and be arranged in a first two-dimensional array with each other, the pressure sensors are located between the support units and a specific side of the support device and are arranged to form a second two-dimensional array. Next, as shown in step 420, when the user supports this particular side, the pressure sensor measures and generates a two-dimensional pressure distribution. Next, as shown in step 430, the two-dimensional pressure distribution is analyzed to calculate the user's body posture. Then, as shown in step 440, the user's body posture is analyzed, and the positions of one or more apophysis position of the user's body on the support device are marked. Finally, as shown in step 450, it is judged whether the occurrence rates of pressure injuries corresponding to all apophysis position are acceptable. For example, they are all lower than the common critical probability value of all apophysis position or less than the respective critical probability values of each bony protrusion, if so, then stop, adjusting the support forces generated by these support units, if not, the supporting force generated by one or more support units is cyclically adjusted until the occurrence rates of corresponding pressure injuries at all apophysis position are acceptable. In other words, when the user is supported by the support device, a two-dimensional pressure distribution between the user's body and a specific side of the support device is measured, the two-dimensional pressure distribution is then analyzed to identify one or more apophysis positions where the user's body is in direct contact with a particular side of the support device (in particular, each apophysis position is at its respective position on a particular side of the support device). Finally, the different support forces applied by the support device to different parts of the user's body are adjusted until the pressure at each bony protrusion directly contacting the specific side of the user's body and the support device is reduced to an acceptable range.

Obviously, the mainstream commercial approach is to directly reduce the pressure on the user's body where the pressure is greater. The specific content of step 410 and step 420 of the present invention is to demarcate the position of one or more apophysis position between the user's body and the support device from the pressure distribution received by various parts of the user's body, it is therefore necessary to obtain a two-dimensional pressure distribution corresponding to the supporting force applied to the user's body by a particular side of the support device. Moreover, the commercial approach is to directly adjust the pressure on some parts, in the present invention, as shown in step 430 to step 450, process the two-dimensional pressure distribution to obtain the global apophysis position position, at the same time, related adjustments are made to reduce the probability of pressure injury at these apophysis position.

At step 430, there are two options for analyzing the two-dimensional pressure distribution to infer the user's body posture: option one is to analyze the two-dimensional pressure distribution and calculate the user's body posture by referring to the user's physiological information. Because the force on the apophysis position is usually not the most stressed part of the user's body, the two-dimensional pressure distribution can only show the pressure on each position on a specific side of the support device, and cannot directly display the position of each apophysis position. Therefore, it is necessary to correspond to the two-dimensional pressure distribution of the user's body according to the specific details of the user's body, and calculate the user's body posture on the support device. Such as lying down, lying down, sitting, lying on the front, lying on the side, lying on the stomach, opening the limbs in a big font, putting the hands on the chest, straightening the legs, putting the hands under the head, sitting cross-legged, kneeling, putting the limbs on the ground, Lying posture, prone posture, side lying posture, hands and feet crossed posture, various body postures. For example, according to whether the user has a prosthetic or auxiliary device and its size profile, the part corresponding to the prosthetic or auxiliary device in the two-dimensional pressure distribution can be excluded, therefore, part of the two-dimensional pressure distribution does not correspond to any apophysis position of the user's body. It can also be based on the user's disability status, for example, if the limbs are incomplete, the possibility of processing is reduced in the process of analyzing which body posture the two-dimensional pressure distribution may correspond to, if the user lacks an arm and does not have a prosthetic, there is no need to consider body posture where both hands touch a specific side of the support device. Furthermore, it can also be based on the user's body shape and the user's disease status, such as whether the user is fat in the waist, abdomen, thighs, buttocks or limbs, or whether the user has edema, sarcoma, fracture, bone bending or joint stiffness and other diseases and the degree of disease When analyzing the body posture corresponding to the two-dimensional pressure distribution, it is more efficient to locate the parts corresponding to the bones in the two-dimensional pressure distribution in the body posture.

On the other hand, the user's height, limb length and weight are the basic physiological information of the user, which can be used to judge that the parts with higher pressure in the two-dimensional pressure distribution should correspond to the same user's body, and exclude those that have nothing to do with the user's body (at least those that have nothing to do with the position of the user's bones) overweight or underweight signal. Alternatively, another option is to analyze the two-dimensional array pressure distribution and infer the body posture with reference to a database model that contains a number of two-dimensional array pressure distributions generated by previous tests and a validated corresponding number of body posture. That is to say, by comparing with a large amount of data, it is possible to find a previous two-dimensional pressure distribution that is closest to the current two-dimensional pressure distribution (or find some previous two-dimensional pressure distributions that are quite close), then with the previous body pose corresponding to this previous two-dimensional array (2D) pressure distribution (or when these previous two-dimensional pressure distributions all correspond to a certain previous body posture), or directly as the current body posture, or as a starting point to calculate the current body posture. Obviously, the former option can accurately deduce the user's body posture according to his personal condition, while the latter option can quickly deduce his body posture.

In block 440, there are four commonly used options on how to analyze the posture of the user's body to demarcate the position of one or more apophysis position of the user's body on the support device. One is to calibrate one or more body parts based on body posture and two-dimensional pressure distribution, then mark the position of one or more apophysis position on the support device according to the physiological information of the user. Example, it is first judged that the larger parts of each pressure in the two-dimensional pressure distribution are those body parts corresponding to the body posture respectively. Then according to various information related to the user's body, the position of one or more apophysis position in each body part with greater pressure is judged. Example, when the body posture is lying on the back, first judge which part of the head, shoulders, buttocks or limb joints corresponds to the greater pressure. Then according to the general human body structure that the occipital bone is approximately in the middle of the head or the like when lying on the back, it is judged that the occipital bone and other apophysis position of the user's body are in the specific position of the support device.

The other is to analyze the body posture first to calculate the position of the body's musculoskeletal on the support device. Graphical calculations are then performed to demarcate the position of one or more apophysis position on the support device. That is, after obtaining the user's body posture, first judge the distribution state of the bones and muscles in the user's body in this body posture based on the general human body structure or even the user's physiological information. Then, according to the two-dimensional pressure distribution, the corresponding position on the support device is converted. Finally, according to the general human body structure or the user's physiological information, the position of each bony protrusion in the support device is demarcated from the position of the skeleton in the support device.

It is also possible to introduce the information that different body postures are prone to pressure trauma at those apophysis positions of the body based on the results of one or more clinical studies. Then, according to the user's body posture and the two-dimensional pressure distribution, the position of one or more apophysis position on the support device is calibrated. For example, thousands of experiments have shown that pressure trauma is particularly likely to occur in the ischia in the semi-recumbent position with the upper body tilted at an angle of 66 degrees, then when the user's body posture is the semi-recumbent position with an upper body inclination of 66 degrees, the position of the ischium of the user's body on the support device is calibrated only from the two-dimensional pressure distribution and the posture of the user's body.

Another option is, first, according to the user's body posture and user's physiological information, converting the user's three-dimensional anatomy into a two-dimensional projection on the plane where these pressure sensors are located. Then compared with this two-dimensional pressure distribution, then mark the position of one or more apophysis position on the support device. For example, based on the specific details of the user's body, to determining how the user's body will be distributed in three-dimensional space with such a body posture, then project onto the support device to obtain a two-dimensional projection parallel to the plane where these pressure sensors are located. Then first find three reference points and calibrate their coordinates in the skeleton part of the two-dimensional projection (because three points form a plane). These reference points are then interconnected to produce datum plat and datum line, and gradually carry out coordinate conversion relative to these three reference points for each bony protrusion point that this body posture will be in direct contact with the support device, and obtain the respective coordinates of each bony protrusion on the support device.

In step 450, how to determine according to the occurrence probability of pressure injury at each apophysis position, adjusting one or more support forces produced by all support units to reduce or eliminate pressure injuries at all apophysis positions, there are two options: one is to compare with one or more medical models first, in order to determine the incidence of pressure injury at each apophysis position and generate decompression strategies sorted according to the probability of occurrence, recirculation separately adjusts one or more support forces generated by one or more support units and applied to different parts of the user's body, until the occurrence rates of pressure injuries corresponding to all apophysis positions are acceptable. For example, they are all smaller than the common critical probability value of all the apophysis position or individually smaller than the respective critical probability values of these apophysis position. That is, after finding the position of each bony protrusion, find out the pressure on each bony protrusion from the two-dimensional pressure distribution (or it can be regarded as the support force received by a certain bony protrusion divided by the area [symbol] bony protrusion), then based on the results of previous medical research, judging whether each bony protrusion is under the influence of such factors as pressure and contour shape, the respective probability of developing pressure injury. Then in order of the probability of occurrence of pressure injury, starting from the apophysis position most prone to pressure injury, gradually lower the rate of pressure injury until all condyles have an acceptable rate of pressure injury.

Another option is, after identifying one or more apophysis positions, to adjust the support force generated by one or more support units. If the occurrence rate of pressure injury corresponding to all apophysis position cannot be made acceptable, then adjust one or more supporting forces generated by one or more support units and exerted on different parts of the user's body, until all apophysis positions correspond to an acceptable rate of pressure injury. That is, trial and error can also be used. If using the computing power of a computer or mobile device, quickly analyze and test a large number of possible configurations in which each support unit applies various support forces to different parts of the user's body, until a certain configuration of struts is found that allows the probability of pressure injury to occur at all condyles to be acceptable.

In step 450, according to the occurrence probability of pressure injury at each condyle to adjust one or more support forces produced by all strut units to reduce or eliminate pressure injuries at all apophysis positions, there are four options: the first option is to first identify one or more apophysis positions whose location corresponds to the highest probability of pressure injury, then adjust the supporting force generated by one or more support units until the corresponding pressure injury occurrence probability at the specific bony protrusion is less than the critical probability value, then proceed in a cycle according to the probability of occurrence of corresponding pressure injury at one or more apophysis position that have not been treated, until the occurrence probability of pressure injury corresponding to all apophysis position is less than the critical probability value. That is to say, based on whether the value is greater than the critical probability value, the risk of pressure injury at each bony protrusion is judged and whether the supporting force applied by the support unit has been adjusted to an acceptable standard, and the bony protrusion with the highest risk Start treatment at each apophysis position, and reduce the probability of pressure trauma at each apophysis position until it is lower than the acceptable critical probability value.

Option 2, when there are M apophysis position, the corresponding pressure injury occurrence rate is greater than zero, only for the N apophysis positions corresponding to their positions with a high probability of pressure injury, by adjusting the pressure generated by one or more support units, the occurrence probability of pressure injury corresponding to the position of the N apophysis position is not lower than the critical probability value, where both M and N are positive integers and M is greater than N. This is because some completed tests have found that in a variety of human postures, the incidence of pressure injury is higher or the degree of occurrence is more severe, often at certain apophysis positions. Although pressure injuries may also occur at other apophysis position, the incidence rate and severity are significantly lower. Therefore, when adjusting, it is only necessary to adjust the few apophysis position where the pressure injury occurs, basically, the occurrence rate of pressure injury at other unadjusted apophysis position can also be reduced to not greater than the critical probability value by the way.

Option 3, when there are M apophysis position corresponding to the position of the pressure injury occurrence rate is greater than zero, the decompression strategy when adjusting the pressure generated by one or more support units, it is to reduce the pressure at a apophysis position corresponding to the position with the highest probability of pressure injury by X1 %, reducing the pressure at a apophysis position corresponding to the second highest probability of pressure injury by X2 %. Until the pressure at a apophysis position with the lowest probability of corresponding pressure injury is reduced by XM %. Here, X1, X2, and up to XM are all greater than zero, and X1 is greater than or equal to X2, and X2 is greater than or equal to X3, so until XM−1 is greater than or equal to XM.

Option 4, when there are M apophysis positions whose position corresponding to the occurrence probability of pressure injury is greater than the critical probability value, the decompression strategy when adjusting the pressure generated by one or more support units is to make the position corresponding to the occurrence of pressure injury The pressure at the bony protrusion with the highest probability is reduced by X1 %, reducing the pressure at a apophysis position corresponding to the second highest probability of pressure injury by X2 %, so until the pressure at a apophysis position with the Nth highest probability of occurrence of pressure injury corresponding to its position is reduced by Xn %. And do not set how much the pressure should be reduced at other apophysis position corresponding to the position where the probability of pressure injury is lower. Here X1, X2, until XN are all greater than zero and X1 is greater than or equal to X2, X2 is greater than or equal to X3, and so on until XN−1 is greater than or equal to XN, where M and N are both positive integers and N is greater than N. Here, these two options are further changes of the previous options, simplifying the repeated testing of various possible configurations of these support units, but directly according to the probability of pressure injury occurring at a plurality of apophysis position. Reduce the pressure on each of these apophysis position proportionally, and the greater the probability of pressure injury, the greater the proportion of pressure received, so that the apophysis position can reduce their risk of pressure injury by adjusting the pressure they receive probability. Of course, such an adjustment method is also based on a number of empirical rules obtained from previous tests, and each variable M, N, X1, X2 . . . XN . . . XM is an adjustable variable.

Because in the existing commercial products, only the pressure on the place with higher pressure is lowered (or the supporting force produced by the support unit corresponding to this place is lowered), however, in the present invention, in order to reduce the occurrence rate of pressure injury at a certain bony protrusion, it will not excessively increase the occurrence rate of pressure injury at other apophysis position. It is to simultaneously adjust the support force generated by one or more support units (or to adjust the pressure on one or more parts of the user's body at the same time), so as to keep the pressure injury at one or more apophysis position at the same time the probabilities are all lower than a common critical probability value or the respective critical probability values of each bony protrusion. That is to say, even if there is only one and a single apophysis position where the probability of pressure injury is greater than the acceptable critical probability value, it is not necessary to adjust only one or more support units closest to the bony protruding. It is still possible to adjust the supporting force exerted by one or more support units farther away from the bony protrusion. After all, with the weight of the user's body unchanged (and even with the total weight of the user's clothing unchanged), the redistribution of the different support forces exerted by each support unit must be considered in order to avoid lowering the pressure on one bony prominence to an acceptable level while increasing the pressure on another to an unacceptable level.

In addition, in step 450, it is first possible to obtain the estimated configuration of these support units through computer simulation calculations, and then adjust these support units accordingly. It is also possible to obtain the desired configuration of these support units by continuously and actually adjusting the configuration values of these support units. In the spirit of the present invention, either approach is acceptable. In particular, a number of previous tests have found that, in general medical applications, usually, it only needs to be adjusted less than five times to obtain the configuration of what kind of supporting force each support unit should exert, so that the probability of pressure injury at all apophysis position is low enough to be acceptable. Therefore, computer simulation or actual adjustment can quickly achieve the final desired result, and the process will not cause negligible side effects on the user's body. One option is to obtain a specific adjusted two-dimensional support force distribution that can make the occurrence probability of pressure injury corresponding to all apophysis position less than the critical probability value by computer simulation, then according to this specific adjustment of the two-dimensional support force distribution to actually adjust the support force generated by support units. Another option is to actually adjust the supporting force produced by these support units after obtaining a specific adjusted two-dimensional supporting force distribution that can make the occurrence probability of pressure injury corresponding to all apophysis position less than the critical probability value. Therefore, when the specific adjusted two-dimensional support force distribution is obtained, the support forces generated by these support units have been adjusted.

In the method for reassigning the body pressure distribution of the support device of the present invention, artificial intelligence can also be used to execute the step 430, the step 440 and/or the step 450. For example, using an artificial intelligence to analyze the two-dimensional pressure distribution to calculate the user's body posture, and through the obtained two-dimensional pressure distribution and the user's body posture and the two-dimensional pressure distribution and the user's body posture obtained in other ways the comparison results of poses are used to train this artificial intelligence. Or use an artificial intelligence to analyze the body posture to demarcate the position of one or more apophysis position of the user's body on the support device, and obtain the position of one or more apophysis position obtained by it and use other methods to obtain the artificial intelligence is trained by comparing the positions of one or more apophysis position. For example, use an artificial intelligence to determine whether the probability of occurrence of pressure injury corresponding to all apophysis position is not greater than a critical probability value and determine how to adjust the supporting force generated by one or more support units so that the pressure corresponding to all apophysis position The occurrence probability of sexual injury is not greater than this critical probability value, and the artificial intelligence is trained by its improvement of pressure injury results for one or more strut unit adjustment results.

The AI can further adjust and optimize by the AI to perform any of the step boxes and comparing the results obtained by the AI with the results obtained by other means, or by using the AI to perform the three step boxes and comparing the stress injury rate obtained by the AI with the stress injury rate that would have occurred if the AI had not been adjusted. For example, when the relationships between various human body types and various dangerous synapses are categorized into several groups after accumulating several cases, the human body types obtained by AI processing can be directly compared with these accumulated cases to obtain the possible dangerous synapses, and the possible dangerous synapses can be feedback and corrected by comparing the based human body types with the possible dangerous synapses identified by AI. Such a correspondence is possible.

As above, another embodiment of the present invention provides a method for redistributing body pressure distribution by a support device, comprising: firstly, providing a support device to support a lying human body, the support device has a plurality of support units and a plurality of pressure sensing units, and the plurality of pressure sensing units are located between the plurality of support units and a lying human body, and by a plurality of the pressure sensing units are continuously monitored during the adjustment procedure, and the plurality of support units are arranged with two-dimensional array, and the different support units can generate respective support forces thereof, and the plurality of pressure sensing units are arranged by each other with two-dimensional array, wherein the initial internal uniform pressure within the plurality of the support units is a specific internal saturated pressure, the specific internal saturated pressure can use the specific value measured by the Shaw hardness tester as a benchmark, and the two-dimensional refers to the directions of the X and Y axes formed by the planes on which the support units are distributed; the layout density of the pressure sensing units is higher than the layout density of the support units. Furthermore, the plurality of support units are arranged with two-dimensional array, and the plurality of pressure sensing units are arranged by each other with two-dimensional array, wherein the distance between at least two pressure sensors is less than 3 cm from each other, and the distance between the centers of at least two pressure sensors is less than 3 cm, and the support units can adjust at least one of the following: horizontal size, vertical size and hardness, and the supporting force within the support unit and the size profile thereof can be changed by the flow quantity of fluid within the support unit.

When a surface of a human body contacts and exerts pressure on the surface of the specific side of the supporting device, performing a measurement step for pressure distribution to scan a lying pressure image of the human body, and measure the lying body pressure of the human body on the support device by a plurality of the pressure sensors, so as to create a first two-dimensional pressure distribution, wherein the first two-dimensional pressure distribution is the vertical pressure caused by the body force at the two-dimensional coordinate position on the position perpendicular to the plane. And then, analyzing the first two-dimensional pressure distribution to generate at least one characteristic parameter, wherein the characteristic parameter comprises boundary profile, number and configuration of regional center of gravity, local peak points of pressure, size and configuration of connecting line between center of gravity and peak value, estimated configuration ratio, and the body condition parameters include measurements, height, weight, and special factors. Then performing a comparison step of the lying posture according to the characteristic parameter and a body type factor to identify a lying posture, wherein the body type factor comprises height, weight, waist circumference and limb defects, and the body type factor can also be obtained from the database of clinical data, further the result of the comparison step of the lying posture can be combined with the wound location information for warning the improper oppression behavior. All the above steps of the present invention can be compared, judged, analyzed and automatically controlled by a artificial intelligence machine learning and big data analysis, wherein the comparison step of the lying posture further comprises an comparison learning by the artificial intelligence to detect the lying posture of user in this situation, and divide into various classifications such as lying on the back, lying on the left side, lying on the right side, lying on the stomach, and whether the hands and feet are crossed. And then, performing a calibration step of apophysis coordinate according to the lying posture and the characteristic parameter to identify the two-dimensional coordinate positions where the important part of musculoskeletal lying on the supporting device, and identify at least one apophysis position of the lying human body and a apophysis coordinates where pressed on the supporting device by the apophysis position. Following, performing a judging step for the probability of pressure injury to detect a pressure at one of the local peaks corresponding to the apophysis coordinate, and determine the probability of pressure injury of the apophysis position to generate a risk degree for each apophysis position, wherein the higher the risk degree means that the higher pressure of the local peak at the apophysis position and the higher probability of pressure injury, wherein the judging step for the probability of pressure injury is to determine whether the support pressure is easy to cause pressure injury based on the type of patients in a clinical research database, and then calculating the probability of pressure injury of the apophysis position and creating the risk degree thereof.

After that a ranking step for the probability of the pressure injury of the apophysis positions is performed by the risk degree to create a risk degree ranking, and then the support force for the apophysis coordinate is recalculated and redistribute by the risk degree ranking, and a parameter of a redistribution model is generated, so that all the support units within the two-dimensional array are redistributed the supporting forces required respectively simultaneously. Then a pressure configuration is generated with the parameter of the redistribution model to proceed a redistribute step for the pressure distribution of the lying human body, so as to reduce the risk degree of the apophysis positions, and the individual shape and hardness of the support unit located at the part in the higher probability of pressure injury can be drove and adjusted according to the pressure configuration, wherein the pressure configuration comprises all the necessary pressure data for regulating the support unit located at the apophysis position with the risk degree, thereby redistributing the support pressure of the support unit when the human body lies on the support device, so as to reduce the pressure at the local peak corresponding to the part in the higher probability of pressure injury.

Additionally, a reducing pressure step for pre-reduce the specific saturated internal pressure is performed according to a pressure reduction percentage and/or a pressure reduction value before the redistribute procedure for the pressure distribution of the lying human body, wherein the parameter of the redistribution model further include the pressure reduction percentage and/or the pressure reduction value, and the pressure reduction percentage is about 5% to 35% of the original saturated internal pressure, and the better is 15% to 25%, which the pressure reduction percentage is the ratio to the previous saturation internal pressure. Finally, the measurement step for pressure distribution is repeat to generate a second two-dimensional pressure distribution, and then the all of above steps are repeat according to the second two-dimensional pressure distribution, wherein if the second two-dimensional pressure distribution shows that the pressure at the local peaks corresponding to the part in the higher probability of pressure injury fails to make the probability of pressure injury at the each apophysis position lower than a predetermined critical probability value, cyclically scan the lying pressure image of the human body and adjust the parameter of the redistribution model in batches by the second two-dimensional pressure distribution until the second two-dimensional pressure distribution shows that the probability of pressure injury at the each apophysis position lower than the predetermined critical probability value, and/or the risk degree is lower than the critical probability value.

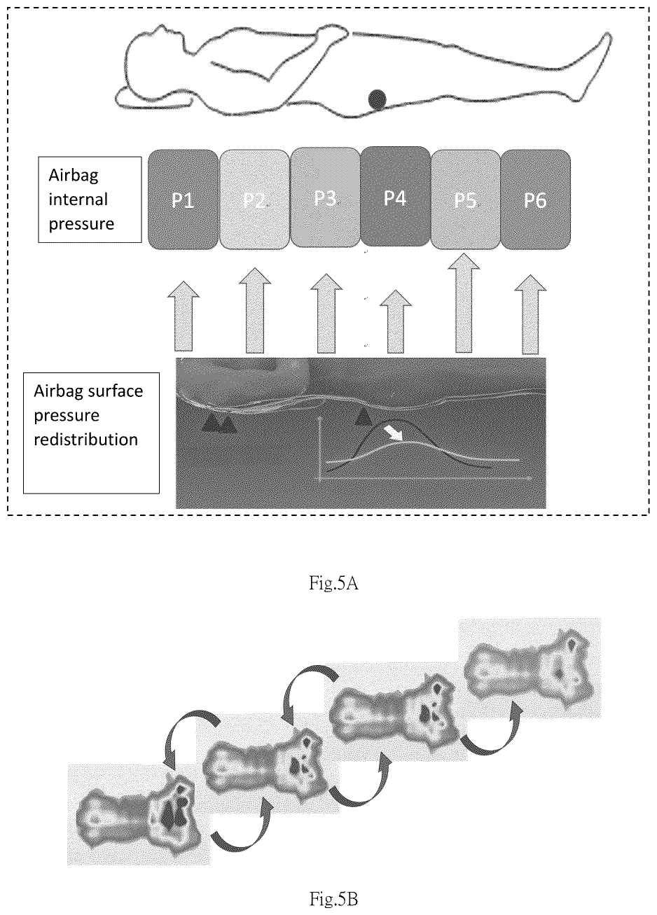

In the present invention, the method for adjusting the pressure distribution is completely different from the method of adjusting a single airbag currently used in the market. The present invention provides the system for redistributing body pressure distribution to find out the optimized overall pressure distribution image and adjust the composition of the corresponding overall airbag pressure accordingly, rather than find out a single pressure point to adjust a single airbag. The system of the present invention redistributes the body pressure distribution to reduce the surface pressure of the airbag at the easily injured part of the whole surface, rather than the internal pressure of the airbag at a single point. The present invention adjusts the internal pressure within the airbag to change the distribution of the supporting force, so that the body surface pressure at the most coordinate positions can be adjusted with the lowest number of airbags, as FIG. 5A, reducing the pressure in the P4 airbag will increase the pressure in the P3 and P5 airbags. In addition, the internal pressure of each airbag will affect the hardness and height shape of each area of the mattress, and the combination of several airbags with different internal pressures will cause different body support pressure distribution images outside the airbags to the body pressure distribution of the reclining person. If there is an increase in support pressure within some parts, there will be a decrease in support pressure within other parts, because the overall weight is unchanged. The present invention can correspond to different pressure distribution images outside the airbag according to various pressure distribution within the airbag, so as to find the optimal model for overall pressure distribution to avoid pressure injury, as FIG. 5B, the present invention can continuously operate the airbag to scan the overall pressure image at the same time (initially as the leftmost pressure distribution image), and then calculate it synchronously and feedback control the internal pressure within the airbag of all support units to continuously optimize the surface pressure, and finally obtain the optimized images of different body support pressure distributions located outside the airbag, such as the pressure distribution image on the far right.

What is claimed is:

1. A method for redistributing body pressure distribution by a support device, comprising:
Providing a support device to support a lying human body, the support device has a plurality of support units and a plurality of pressure sensing units, and the plurality of pressure sensing units are located between the plurality of support units and a lying human body, and by a plurality of the pressure sensing units are continuously monitored during the adjustment procedure, and the plurality of the support units are arranged in one or a plurality of groups of the two-dimensional array, and the different support units can generate respective support forces thereof, and a plurality of the pressure sensors are arranged by each other to form one or a plurality of groups of the two-dimensional array, wherein the initial internal uniform pressure within the plurality of the support units is a specific internal saturated pressure, and the two-dimensional refers to the directions of the X and Y axes formed by the planes on which the support units are distributed;
when a surface of a human body contacts and exerts pressure on the surface of the specific side of the supporting device, performing a measurement step for pressure distribution to scan a lying pressure image of the human body, and measure the body of the lying pressure of the human body on the support device by a plurality of the pressure sensors, so as to produce a two-dimensional pressure distribution, wherein the two-dimensional pressure distribution is the vertical pressure caused by the body force at the two-dimensional coordinate position on the position perpendicular to the plane ;
analyzing the two-dimensional pressure distribution to generate at least one characteristic parameter;
Performing a comparison step of the lying posture according to the characteristic parameter and a body type factor to identify a lying posture;
performing a calibration step of a apophysis coordinate according to the lying posture and the characteristic parameter to identify the two-dimensional coordinate positions where the important part of musculoskeletal lying on the supporting device, and identify at least one apophysis position of the lying human body and a apophysis coordinates where pressed on the supporting device by the apophysis position;
performing a judging step for the probability of pressure injury to detect a pressure at one of the local peaks corresponding to the apophysis coordinate,
and determine the probability of pressure injury of the apophysis position, to generate a risk degree for each apophysis position, wherein the higher the risk degree means that the higher the pressure of the local peak at the apophysis position and the higher the probability of pressure injury;
performing a ranking step for the probability of the pressure injury of the apophysis positions by the risk degree to generate a risk degree ranking, recalculating and distribute the support force for the apophysis coordinate by the risk degree ranking, and generate a parameter of a redistribution model, so that all the support units of the two-dimensional array in the plurity of group are redistributed the supporting forces required respectively simultaneously;
generating a pressure configuration with the parameter of the redistribution model to proceed a redistribute procedure for the pressure distribution of the lying human body; and
driving and adjusting the individual shape and hardness of the support unit located at the part in the higher probability of pressure injury according to the pressure configuration, wherein the pressure configuration includes all the necessary pressure data for regulating the support unit located at the apophysis position with the risk degree, so as to redistribute the support pressure of the support unit when the human body lies on the support device, therefore reduce the pressure at the local peaks corresponding to the pressure injury with high risk.

2. The method according to claim 1, wherein the body type factor comprises height, weight, waist circumference and limb defects, and the body type factor can also be obtained from the database of clinical data.

3. The method according to claim 1, further comprises a artificial intelligence machine learning and big data analysis to compared, judged, analyzed and automatically controlled for all the step, wherein the comparison step of the lying posture further comprises an comparison learning by the artificial intelligence to deduct the lying posture of user in this situation, and divide into various classifications such as lying on the back, lying on the left side, lying on the right side, lying on the stomach, and whether the hands and feet are crossed.

4. The method according to claim 1, further comprises a reducing pressure step for pre-reduce the specific saturated internal pressure according to a pressure reduction percentage and/or a pressure reduction value before performing the redistribute procedure for the pressure distribution of the lying human body, wherein the parameter of the redistribution model further include the pressure reduction percentage and/or the pressure reduction value, and the pressure reduction percentage is about 5% to 35% of the original saturated internal pressure, and the better is 15% to 25%, which the pressure reduction percentage is the ratio to the previous saturation internal pressure.

5. The method according to claim 1, further comprises repeating the measurement step for pressure distribution to generate an updated two-dimensional pressure distribution, and than repeating the above steps according to the updated two-dimensional pressure distribution, wherein if the two-dimensional pressure distribution shows that the pressure at the local peaks corresponding to the part in the higher probability of pressure injury fails to make the probability of pressure injury at the each apophysis position lower than a predetermined critical probability value, cyclically scan the lying pressure image of the human body and adjust the parameter of the redistribution model in batches by the updated two-dimensional pressure distribution until the updated two-dimensional pressure distribution shows that the probability of pressure injury at the each apophysis position lower than the predetermined critical probability value, and/or the risk degree is lower than the critical probability value.

6. The method according to claim 1, further comprises at least one selected from the following consisting of:
the layout density of the pressure sensors is higher than the layout density of the support units;
the pressure sensors are arranged in a two-dimensional array; and
the support units are arranged in a two-dimensional array.

7. The method according to claim 1, further comprises at least one selected from the following consisting of:
the distance between at least two pressure sensors is less than 3 cm from each other; and
the distance between the centers of at least two pressure sensors is less than 3 cm.

8. The method according to claim 1, wherein the support units can adjust at least one of the following: horizontal size, vertical size and hardness, the supporting force within the support unit and the size profile thereof can be changed with the flow quantity of fluid within the support unit.

9. A method for redistributing body pressure distribution by a support device, comprising:
providing a support device, said support device has a plurality of support units and a plurality of pressure sensors, said pressure sensors are located between said support units and a specific side of said support device, said support units are arranged with each other to form a first two-dimensional array and different said support units can generate respective supporting forces, said pressure sensors are also arranged with each other to form a second two-dimensional array;
when a user is supported on this particular side, said *pressure sensors are used to measure and generate a two-dimensional pressure distribution;
analyzing the two-dimensional pressure distribution, and calculate a body posture of the user;
analyzing the body posture, demarcating the positions of at least one apophysis position of the user's body on said support device; and
determining if the incidence of pressure damage is acceptable for all apophysis positions, and if so, stop adjusting the support force generated by said support units, and if not, adjust the support force generated by one or more said support units in a cyclic manner until the incidence of pressure damage is acceptable for all said apophysis positions.

10. The method according to claim 9, further comprising:
the distribution density of the pressure sensors is higher than the distribution density of said support units;
said *pressure sensors are arranged in a two-dimensional array;
said support units are arranged in a two-dimensional array;
the distance between at least two said *pressure sensors is less than three centimeters from each other;
the distance between the centers of at least two said *pressure sensors is less than three centimeters;
at least one said support unit can adjust at least one of the following: horizontal size, vertical size and hardness;
at least one said support unit can change the support force generated by it by changing the fluid inside it; and
at least one said support unit can change its dimensional profile by changing its internal fluid.

11. The method according to claim 9, further comprising:
all said support units generate the same support force before the user is supported by the said support device; and
before the user is supported by the said support device, at least two said support units generate different support forces.

12. The method according to claim 9, further comprising:
analyzing the two-dimensional pressure distribution and calculate the body posture with reference to the user's physiological information, the user's physiological information includes at least one of the following: the user's height, the user's weight, the length of the user's limbs, the user's body shape, the user's disability status, the user's disease status, the size profile of the prosthetic limb used by the user, and the size profile of the assistive device used by the user;
referring to the database model to analyze the two-dimensional pressure distribution and calculate the body posture, the database model contains a plurality of two-dimensional pressure distributions generated by previous tests and a plurality of corresponding body postures verified;
Calibrating one or more body parts based on body pose and two-dimensional pressure distribution, then mark the position of one or more said apophysis position on said support device according to the user's physiological information, wherein, the body posture further includes one of the following: lying posture, prone posture, side lying posture, and hands and feet crossed posture, and the user's physiological information includes one of the following: the user's height, the user's weight, the user's body shape, the user's disability status, the user's disease status, the size profile of the prosthetic limb used by the user, and the shape of the assistive device used by the user size profile;
Analyzing the body posture to calculate the position of the body's musculoskeletal on said support device, then perform graphical calculations to demarcate the position of at least one said apophysis position on said support device;
introducing different body positions according to one or more clinical studies, separately, a message of stress trauma prone to said apophysis positions of the body, then according to the user's body posture and the two-dimensional pressure distribution to calibrate the position of one or more apophysis position in said support device; and
according to the user's body posture and user's physiological information, to convert the user's three-dimensional anatomy into a two-dimensional projection on the plane where said *pressure sensors are located, then compared with this two-dimensional pressure distribution, then mark the position of at least one said apophysis position on said support device.

13. The method according to claim 9, further comprising:
determining whether all apophysis positions correspond to the incidence of pressure injury, all acceptable standards are whether all apophysis positions correspond to the occurrence rate of pressure injury, no greater than the common critical probability value of these apophysis position;
determining whether all said apophysis positions correspond to the incidence of pressure injury, the acceptable standard is whether each said apophysis position corresponds to the occurrence rate of pressure injury, no greater than their respective critical probability values;
comparing with one or more medical models, in order to judge the incidence of pressure injury at each said apophysis position, and generate decompression strategies sorted according to the probability of occurrence, cyclically adjust the supporting force generated by one or more said support units, up to the respective rates of pressure injury at all said apophysis position are acceptable; and
after identifying one or more said apophysis positions, to adjust the supporting force generated by one or more said support units, if the occurrence rates of pressure injuries corresponding to all said apophysis position cannot be made acceptable, then cyclically adjust the supporting force produced by at least one said support units, until all said apophysis positions correspond to an acceptable rate of pressure injury.

14. The method according to claim 9, further comprising:
Identifying the specific synapse or synapses with the highest incidence of pressure injury in relation to its location, and then adjust the support force generated by at least one said support units until the specific synapse or synapses have an acceptable incidence of pressure injury, and then cycle through the other synapses or synapses that have not yet been treated in order of their corresponding incidence of pressure injury until all synapses have an acceptable incidence of pressure injury;
finding the corresponding position in one or more said apophysis positions, which a specific said apophysis position where the risk of pressure injury is greatest, then to adjust the supporting force generated by one or more said support units until the probability of occurrence of pressure injury corresponding to the specific said apophysis position is acceptable, then proceed in a cycle according to the probability of occurrence of corresponding pressure injury at said apophysis position that have not been treated, until the corresponding pressure injury occurrence rate at all said apophysis position is acceptable;
when the position-related compressive injury probability at M said apophysis positions is greater than zero, only the N said apophysis positions with a greater position-related compressive injury probability are adjusted by adjusting the pressure generated by one or more support units so that the position-related compressive injury probability at each of the N said apophysis position s is not greater than the critical probability value, where M and N are positive integers and M is greater than N;
when the occurrence probability of pressure injury corresponding to the position of M said apophysis position is greater than zero, decompression strategy when adjusting the pressure generated by one or more strut elements, which is to reduce the pressure at said apophysis position corresponding to the position with the highest probability of pressure injury by X₁ %, reducing the pressure at a apophysis position corresponding to the second highest probability of pressure injury by X₂ %, until the pressure at said apophysis position with the lowest probability of corresponding pressure injury is reduced by X_{M} %, here X₁, X₂, until XM are not less than zero and X1 is greater than or equal to X₂, X₂ is greater than or equal to X₃ so until X_{M-1} is greater than or equal to X_{M}; and
when there are M said apophysis position, the corresponding pressure injury occurrence rate is greater than zero, decompression strategy when adjusting the pressure generated by one or more strut elements, which is to reduce the pressure at said apophysis position corresponding to the position with the highest probability of pressure injury by X₁ %, reducing the pressure at said apophysis position corresponding to the second highest probability of pressure injury by X₂ %, until the pressure at said apophysis position with the Nth highest probability of occurrence of pressure injury corresponding to its position is reduced by Xₙ %, and for other said apophysis positions with lower incidence of pressure injury corresponding to its position, there is no setting how much the pressure should be reduced, here X₁, X₂, until XN are all greater than zero and X₁ is greater than or equal to X₂, X₂ is greater than or equal to X₃ so until X_{N-1} is greater than or equal to X_{N}, where M and N are positive integers and M is greater than N.

15. The method according to claim 9, further comprising:
first obtains a specific adjusted two-dimensional support force distribution by computer simulation that results in an acceptable incidence of pressure damage to all said apophysis position, and then actually adjusts the support force generated by one or more said support units according to this specific adjusted two-dimensional support force distribution; and
actually adjusting the support forces generated by said support units after obtaining a specific adjusted two-dimensional array support force distribution that results in an acceptable incidence of pressure damage to all of said apophysis positions, so that the support forces generated by said support units are adjusted when this specific adjusted two-dimensional array support force distribution is obtained.
